(19) United States
(12) Patent Application Publication
(10) Pub. No.: US 2020/0261189 A1
(43) Pub. Date: Aug. 20, 2020

(54) IMPLANT SHAPED TO BE ADAPTED TO BONE STRUCTURE COMPRISING A BASE AND ASSOCIATED PRODUCTION METHOD

(71) Applicant: Karl Leibinger Medizintechnik GmbH & Co. KG, Muhlheim (DE)

(72) Inventors: Axel WAIZENEGGER, Mühlheim (DE); Frank REINAUER, Mühlheim (DE); Nils-Claudius GELLLRICH, Mühlheim (DE); Bjorn RAHLF, Mühlheim (DE)

(21) Appl. No.: 16/062,783

(22) PCT Filed: Nov. 30, 2016
(86) PCT No.: PCT/EP2016/079244

§ 371 Date: Jun. 15, 2018

(30) Foreign Application Priority Data
Dec. 23, 2015 (DE) 10 2015 122 800.9

(51) Int. Cl. A61C 8/00; A61C 13/00; A61B 6/14; A61B 6/03; A61B 5/055; A61B 5/00; G05B 19/4099

(52) U.S. Cl. CPC A61C 8/0031; A61C 8/005; A61C 13/0004; A61B 6/14; A61B 6/032; G05B 2219/35012; A61B 5/0035; A61B 5/0036; A61B 5/004; G05B 19/4099; A61B 5/055

(57) ABSTRACT

The invention relates to an implant for attaching to a bone with a support structure which comprises at least one securing portion which follows the bone outer structure and is to be attached to the bone, wherein a base for receiving a prosthesis directly or by using an intermediate part (abutment) projects from the support structure. In addition, the invention also relates to a method for producing an implant, comprising the step of capturing individual patient data, and creating the support structure and/or the base on the basis of the individual patient data.

The present invention relates to an implant for attaching to a bone, for example of a mammal such as a primate, e.g. a human being, with a support structure which comprises at least one securing portion which follows the bone outer structure for being attached to the bone, and to a method for producing such implant.

From prior art an implant which is used, inter alia, as a jaw implant is known already. Said implant has a substantially plate-shaped configuration which can be adapted to the contour of the bone merely to a restricted extent in a maximum of two spatial planes.

The plate-shaped implant is most largely adapted to the bone structure so that the implant at the best rests and, resp., abuts on the same, i.e. abuts in one plane only, and is fastened to the bone structure by a screw. The positional stability of said implant is thus realized via bearing or abutting areas of the implant on the bone structure and a screw, with the position of the screw being predefined by the implant structure.

For positioning the implant during operation, multiple checking of the positioning of the implant is required. For this purpose, separate positioning aids are used, thus requiring the operating surgeon to change his/her grip.

Since the shaping of the implant can be adapted to the bone structure to a limited extent only, a permanently stable positioning via abutting and, resp., bearing areas cannot be ensured. Securing of the entire implant by means of one single screw is capable of preventing merely a shifting transversely to the screwing axis. However, the screwed connection may get loose over time at least so far that possible rotation of the implant about the screwing axis cannot be permanently prevented.

Moreover, the position of the securing (of the screw) is defined already by the predetermined geometry of the implant. This results in the fact that the implant possibly cannot be inserted, as in the area of securing nerves and/or existing teeth are present which are damaged when the implant is secured by means of the screw. In case that existing intact teeth obstruct the securing of the implant, such teeth have to be equally removed and have to be replaced with artificial teeth.

In addition, the use of said implant requires an existing intact bone structure. Unless the latter is available, the bone structure has to be reconstructed in advance, for example by replacing the missing structures by bones from the iliac crest or from the fibula of the patient. For this, hospital treatments of the patient are necessary.

The implant includes structures such as bores having a female thread which are prepared to receive one (or more) so called abutment bases. The latter are usually screwed into the structures provided for this purpose. An abutment base in turn is prepared for receiving a so-called abutment which serves for receiving an artificial dental prosthesis. This means that the dental prosthesis is connected to the implant via abutment bases and abutments.

The already existing structures prepared for receiving the abutment bases fixedly predetermine the alignment of the abutment bases—and thus also of the abutments. In this way, the alignment of the dental prosthesis is defined and can be adapted only slightly to the individual dentition structure of the patient.

Moreover, the screwing axis of the abutment base into the implant usually also corresponds to the screwing axis of the abutment into the abutment base. In this way, the implant cannot be adapted (or can only be adapted to a restricted extent) to the flux of force resulting from the chewing motion. This results in premature fatigue symptoms of the implant material culminating in fatigue fractures.

It is the object of the invention to eliminate or at least alleviate the drawbacks of prior art, and especially to provide an implant which is adapted to be implanted even without any preceding reconstructions of the bone while avoiding major surgical interventions as well as to provide a method for producing such implant as well as a method for implanting said implant.

The object of the invention is achieved by the fact that a base for receiving a prosthesis, such as an endoprosthesis or an exoprosthesis, projects from the support structure directly or using an intermediate part (abutment).

Advantageous embodiments are claimed in the subclaims and shall be explained hereinafter.

It is of advantage when the base is in the form of an elevation or prominence standing out against the ambient outer contour of the support structure, for example of the type of projection. Thus, further components can be quickly and permanently fastened to or in or via said base at a predetermined position.

In addition, it is of advantage when the base is an integral, single-piece and preferably single-material component of the support structure. The integral formation of the support structure and the base helps to avoid a junction and thus a potential weak point. As a result, an especially stable implant is obtained.

Another aspect of the invention provides the base to be prepared for non-positively, positively and/or adhesively receiving the prosthesis or an intermediate part. This enables simple connection or mounting of the prosthesis or of the intermediate part.

An advantageous embodiment provides that the preparation is a thread such as a female or male thread or a retention shape, i.e. such contour which facilitates or enables positive and/or non-positive securing. The retention shape advantageously incorporates an undercut.

It is moreover advantageous when the retention shape includes a dome-shaped, ball-shaped or spherical distal part. The distal part enables simple attachment to or simple connection to the prosthesis or the intermediate part.

It is further advantageous when the base has a snap-fit design. In this way, the prosthesis or the intermediate part can be easily clipped to the base and any further connecting elements such as e.g. screws can be dispensed with.

Another advantageous embodiment provides that the intermediate part is designed as a dental implant and holds, preferably while interposing an abutment, an artificial tooth or a crown or is prepared for holding or takes the shape of an abutment.

It is of advantage that the base extends along a direction transversely or diagonally to a longitudinal extension direction of the support structure. This enables the inclination of the base to correspond to the inclination of an artificial tooth/a crown secured thereto. Thus, adaptation of the artificial denture to the individual dentition structure of the patient—and hence also a flux-optimized positioning of the prosthesis-implant combination—is possible.

Moreover, it is of advantage when plural bases designed in the manner of posts are present. Such structure enables plural artificial teeth and/or crowns or beam-shaped intermediate parts to be received which, in the form of cross-beams resting on the bases, interconnect all bases so as to improve the mounting and, resp., connection of the prosthesis to the implant and/or to increase the strength of the mount or connection between the prosthesis and the implant.

Another possible advantageous embodiment provides all longitudinal axes of the bases to extend transversely or diagonally to the longitudinal extension direction of the support structure. This allows for aligning each of the bases for each patient individually adapted to the optimum positioning of the prosthesis.

It is also advantageous when all of the longitudinal axes of the bases point exactly to the same spatial direction. In this way, the bases can be connected more easily to a cross-beam so as to enlarge the bearing surface of the prosthesis and/or to improve the stability of the seat of the prosthesis.

Another aspect of the invention provides that the support structure is grid-shaped or has one or more grid portion(s) and/or perforated lands. On the one hand, material and thus also costs can be saved and, at the same time, growing of bone and/or soft tissue into the grid structure can be promoted, which results in a stable connection by formation of a tertiary stability between the implant and the bone surrounding the implant.

It is of advantage in this context that the support structure, the grid portion and/or the land include(s) one perforation or more perforations in the form of a through-hole such as a bore. Thus, the grid structure at the same time may be used as a securing device and consequently separately provided securing points/devices can be dispensed with.

It is of advantage that the through-hole is designed to receive a screw to be screwed into the bone. Thus, the arrangement of separate through-holes on the implant for receiving screws can be dispensed with.

Moreover, it is advantageous to separate or space the distal part from a truncated cylinder portion via a tapered area, as in this way already very small heights between the dental implant and the prosthesis resting thereon can be realized, because no minimum lengths such as a minimum thread depth have to be observed.

It is also advantageous when the base includes a cylindrical outer contour or a flux-optimized outer contour. This helps to avoid fatigue symptoms of the implant material due to a design of the base which is not flux-optimized.

An advantageous embodiment provides that the base has an at least partial hollow-cylindrical shape preferably on the distal side. This shape offers the maximum variation for the configuration of the connection of the prosthesis.

The base or bases is/are advantageously inserted and/or positioned so as to replace bone material and, resp., can be positioned so as to replace bone material. In this way, complex bone reconstructions by one's own or foreign bone material can be avoided.

Another aspect of the invention provides that at an implant plural bone form-locking portions are provided and are geometrically configured and aligned so that a form-locking seat on the bone is enforced, especially during insertion or in the inserted state in the animal or human body. This allows to unambiguously place the implant without any major effort and to dispense with any separate positioning aids.

It is of advantage when the bone form-locking portions are configured and aligned geometrically so that the seat enforces one single stable bearing position of the implant on the bone. Thus, positioning is facilitated and the risk of wrong positioning of the implant is significantly reduced or almost completely avoided.

An implant on which at least three spatially separated bone form-locking portions are present has turned out to be advantageous. Plural spatially separated bone form-locking portions help to increase the abutting and positioning accuracy of the implant.

It is of further advantage when each bone form-locking portion is prepared in a different spatial direction at a different bone portion for abutting against each other. Thus, the abutting and positioning accuracy of the implant is further increased and the positional stability of the implant is enhanced. This means that the implant is largely prevented from shifting in its position.

Moreover, the preparation of the bone form-locking portion is advantageous so that a bone portion can be encompassed. Encompassing of a bone portion helps to further reduce the risk of shifting of the implant.

One advantageous embodiment provides that the bone form-locking portion is formed by the support structure or a component separate therefrom, preferably in one single piece, integrally and/or from one single material. The single-part design of the bone form-locking portion and the support structure help to reduce the number of parts and to save material costs. Moreover, the single-piece design also helps to increase the positioning accuracy of the support structure and/or of the separate component.

The patient-specific tailored design of the bone form-locking portion and/or of the support structure as a solid component such as a rod and/or by an outer contour close or adjacent to the bone by an individual bone and the use of CAD/CAM with respect to the same, has turned out to be advantageous. In this manner, an implant individually adapted to the needs of each patient can be produced.

It is moreover advantageous that in the bone form-locking portion at least one screw seating hole or plural screw seating holes are present. Thus, the bone form-locking portions at the same time serve as a boring template and as a securing device for securing the implant to the bone.

Another advantageous embodiment shows that the bone form-locking portion and/or the support structure includes one or more coupling area(s) so as to fix the bone form-locking portion to the support structure.

Moreover, the presence of multiple grid fastening points has turned out to be of advantage. Thus, the securing of the implant to the bone can be individually adapted to the patient and nerve paths as well as possibly existing teeth can be avoided during securing.

Further advantageous is an implant in which securing areas are predefined and geometrically prepared on the support structure for receiving one or more screws to be screwed into the bone, with one or more bases for receiving a prosthesis being present at a spatial distance thereof. Thus, the support structure serves both as boring and positioning template. In addition, the spatial separation of the mounting of the implant on the bone (first screwing axis) and of the mounting of the prosthesis (second screwing axis) prevents premature fatigue symptoms of the implant material due to excessive mechanical load at one spot.

In this context it is advantageous when a longitudinal axis across the screw to be inserted or being inserted is aligned transversely, diagonally or skew relative to a longitudinal axis of the base, especially a screwing axis of the base. In this way, the direction of the screw being inserted or to be inserted can be set in a flux-optimized manner according to possibly present influencing factors such as nerve paths or teeth and, moreover, the already afore-described local mechanical excessive load of the implant can be avoided.

Another advantageous embodiment provides that the securing area is distanced from the base by more than the length of a screw and/or more than 1.2, 2 or 3 times the thickness in the securing area and less than 500 times the length of a screw and/or less than 400 times the thickness in the securing area. Thus, the required strength of the implant can be ensured and premature fatigue symptoms can be avoided.

It has turned out to be advantageous that the base is configured so that it enables connection of a prosthesis or an intermediate part according to the locking or non-locking principle.

Furthermore, it is of advantage when the implant, for example the support structure and/or the base or one of the bases is in the form of a reservoir for a medical drug or a pharmacological drug. Thus, it is possible to place e.g. drugs, especially those which have to be administered/taken over a quite long period, there in the form of a drug-release system such as a drug-release capsule, and to administer them in this way. This is advantageous especially for patients who permanently have to take medical or pharmacological drugs, as such intake can no longer be forgotten and overdosing can be avoided. In this context, it is obvious to make use of a probe measuring technique.

A further advantage is constituted by preparing the implant for converting chewing energy and preferably for charging accumulators. The energy obtained in this way can be used to supply energy to smaller accumulators present in the body, for example.

Another advantageous embodiment provides the implant to be in the form of a jaw implant, such as a mandibular or maxillary implant. Such implant can be used for partially toothed as well as toothless jaws.

Moreover, it is of advantage when the support structure is designed/prepared in terms of material and geometry so as to enable a telescoping arrangement of the prosthesis. Thus, also states of major bone defects, such as e.g. after tumor operations including the resection of parts of the jaw, can be treated.

It is also advantageous when the support structure and/or the base is/are provided with a coating which promotes bony growth, strengthens the immunologic system, causes an antibiotic effect and/or assumes a reservoir function, for example using bone morphogenetic proteins (BMPs).

In addition, it is of advantage when one component or all components is/are made from titanium, a titanium alloy or a Ti—Al alloy. Titanium and titanium alloys have high bio-compatibility and high inertia and therefore are suited as a material for an implant.

Another advantageous embodiment provides a positioning aid being present on the support structure and/or the base. Said positioning aid assists the operating surgeon during insertion of the implant in checking the correct positioning and subsequently during follow-up in checking whether the implant might have shifted.

In this context, it is advantageous when the positioning aid is a marker, such as a laser marker and/or prominence, e.g. a bead. The prominence is of advantage especially for later checking by means of X-ray, as said prominence is evident from such pictures.

The design of the support structure as a resecting, positioning and/or boring template is advantageous. By integrating said functions in the implant and, resp., the support structure additional means which usually serve as such templates can be dispensed with.

It is of further advantage when the coupling area or the coupling areas include(s) a hole partially or completely penetrating the same, e.g. in the form of a bore, preferably for receiving a screw.

Furthermore, a method of producing an implant is described, comprising the step of capturing individual patient data, including e.g. the bone and/or soft-tissue configuration comprising the respective outer contour, e.g. using MRT or CT, creating the support structure and/or the base on the basis of the individual patient data, e.g. by CAD/CAM techniques, preferably making use of laser-sintering.

Moreover, a method of implanting an implant produced as described above into an animal or human body is described. Modifications which are configured as follows are especially useful:

Thus, it is of advantage when the implant includes screw holes aligned so that they can be used as a boring template for introducing bores into the bone. Hence the implant simultaneously serves as a boring template, and therefore the operating surgeon need no longer position any separate boring template. This helps to considerably facilitate the production of bores in the bone which serve for receiving screws, for example.

It is of advantage in this context when the screw holes are aligned at least diagonally/transversely or skew. Diagonal alignment of the screw holes means here that the screws are not arranged in parallel in one spatial direction, and skew describes the non-parallel alignment of the screw holes in at least two spatial directions. Thus, the screws for securing the implant to the bone can be individually adapted to each patient and can be provided so that neither nerves nor teeth/roots are damaged.

One advantageous embodiment provides that the inner diameter of the screw hole is adjusted to the outer diameter of the drill and/or of the intended hole within the bone. Hence the implant simultaneously serves as a boring template.

It is of advantage when the inner diameter of the screw hole is about 0.8, 0.85 or 0.9 to 0.99 times the intended bone hole. Within this range, precise positioning of the bone hole above the bone hole present in the implant is possible.

Another advantageous embodiment provides that the screw hole is arranged in the area of a support structure of the implant.

It is of advantage when the screw holes are formed to be inclined/diagonal relative to the surface of the support structure. Thus, the individual positioning of the screws according to the respective patient data is possible, while, at the same time, flux-optimized positioning of the screw holes can be provided.

Moreover, it is of advantage when the implant itself is in the form of and, resp., usable as a boring template including drill guide bushes. Thus, the use of a separate boring template can be renounced, with the correct positioning and drilling of the bone holes during surgical intervention being facilitated for the operating surgeon.

Another advantageous embodiment provides that the support structure includes such outer contour, e.g. by extensions, prominences and/or recesses, which results in visible plastic modifications at the person to which the implant is implanted. In this way, plastic corrections and, resp., a reconstruction of originally present contours may be carried out simultaneously with the setting of the implant.

Furthermore, a method of producing such implant is described.

For the method of producing such implant it is of advantage when, on the basis of previously obtained patient-specific data, the screw seating holes are introduced in such manner that after having implanted the implant the screw seating holes are used as a forced guide for a drill which is usable to introduce holes into the bone.

Moreover, a possible embodiment of the implant provides that the implant 1 includes a numbering of the screw seating holes as well as two markers which are provided at the respective left and right ends of the bone contour portions.

The numbering of the screw seating holes serves as an orientation aid for the operating surgeon, because not all of the screw seating holes are used to secure the implant to the bone by means of screws. During the surgical intervention the operating surgeon can see from the numbering into which of the screw seating holes screws are to be set and he/she can check whether he/she has set all screws required.

The markers are laser markers and/or prominences that can be detected by means of a probe. The operating surgeon can check, on the one hand, the correct positioning of the implant via such markers. On the other hand, said markers may be used to mark the area in which the bone has to be resected (e.g. by reason of tumor tissue) and thus during intervention serve as check markers which the operating surgeon can scan with the aid of a probe, and hence he/she can check whether he/she has completely resected the bone area to be removed.

In other words, the invention consists of an implant which serves as a support structure and provides coupling areas for a prosthesis and of a method of producing said implant as well as a method of implanting said implant into an animal or human body.

Hereinafter, the invention shall be illustrated in detail by way of drawings showing different variations, wherein

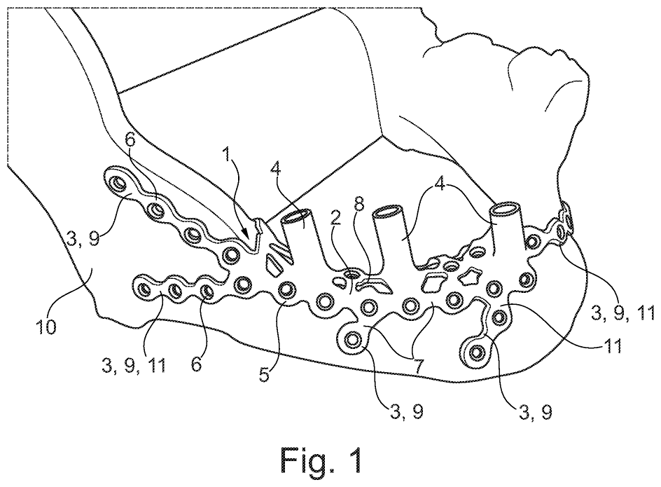

FIG. 1 shows a spatial representation of the implant in a first embodiment, for the mandible,

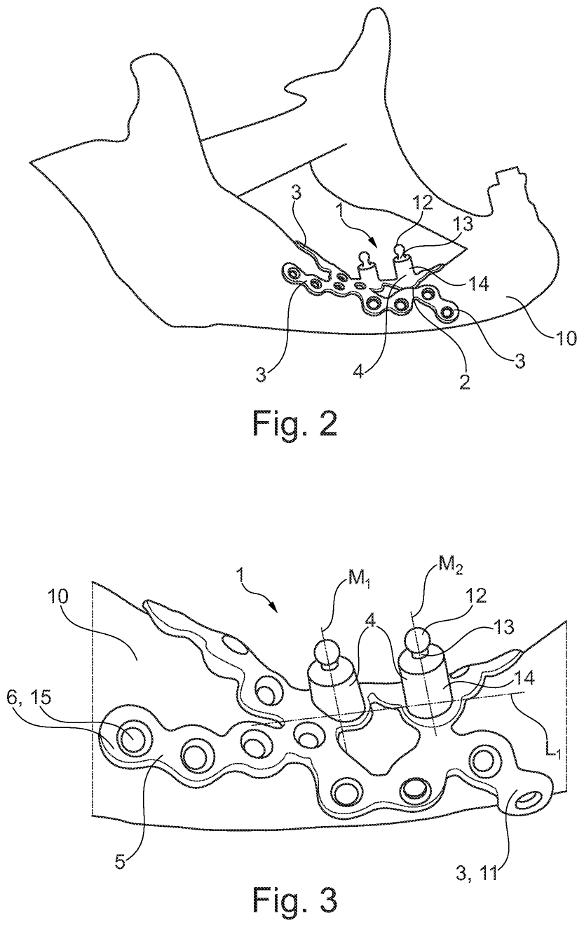

FIG. 2 shows a spatial representation of the implant in a second embodiment, for the mandible,

FIG. 3 shows an enlarged spatial representation of the implant of the second embodiment, for the mandible,

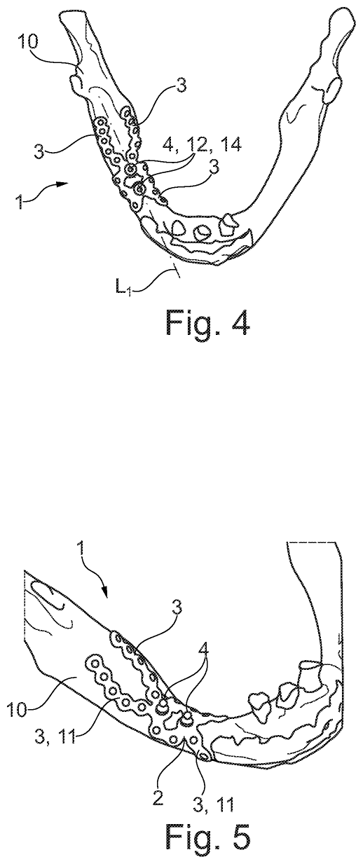

FIG. 4 shows a spatial representation of the implant in a third embodiment, for the mandible when viewed from the top,

FIG. 5 shows a spatial representation of the implant of the third embodiment, for the mandible from a diagonal lateral direction,

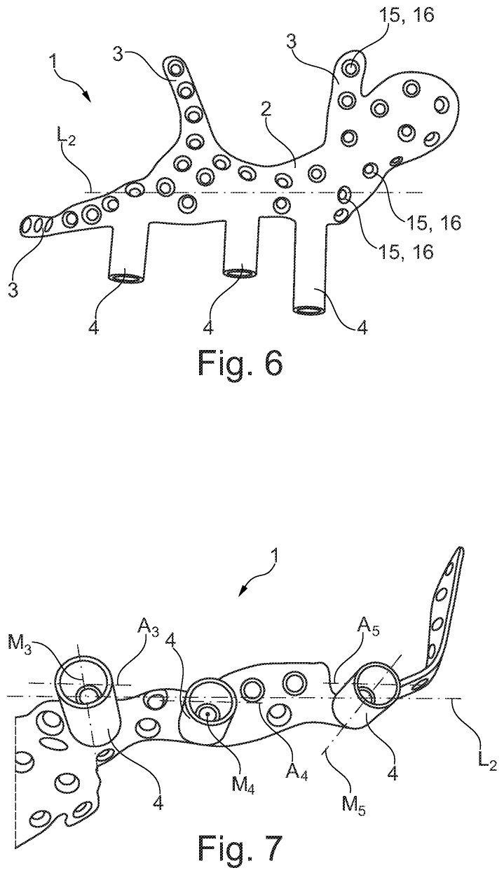

FIG. 6 shows a front view of the implant in a fourth embodiment, for the maxilla,

FIG. 7 shows a top view of the implant of the fourth embodiment, for the maxilla,

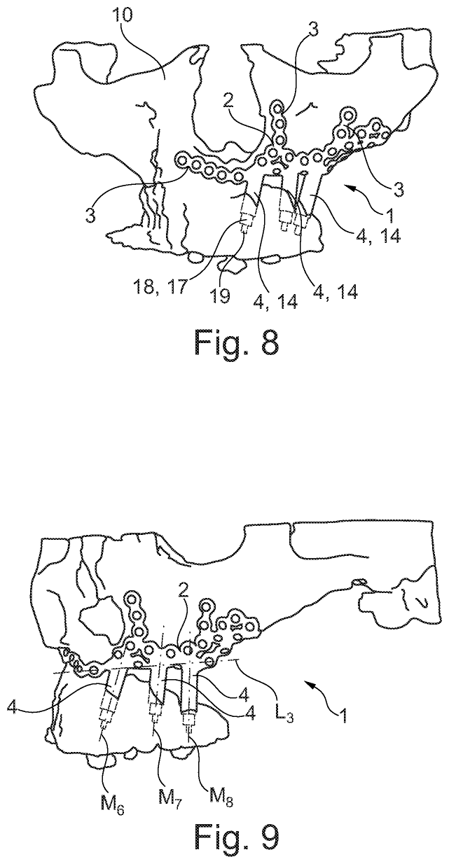

FIG. 8 shows a spatial representation of the implant in a fifth embodiment, for the maxilla in the implanted state, and

FIG. 9 shows a side view of the implant of the fifth embodiment, for the maxilla in the implanted state.

The figures are merely schematic and only serve for the comprehension of the invention. Like elements are provided with like reference numerals. Features of the individual embodiments may also be realized in other embodiments. Consequently, they are interchangeable.

FIG. 1 illustrates a spatial representation of the implant in a first embodiment for the mandible. The implant 1 consists of a support structure 2 including plural securing portions 3 following the bone outer structure and plural bases 4 formed integrally with the support structure 2.

The support structure 2 has a grid-like structure 5 consisting of annular portions 6 which are interconnected via lands 7 of different lengths. The support structure 2 is accurately fitted to the bone contour on which it abuts and, resp., bears, and it can be subdivided into a main body 8 and distally extending secondary bodies 9, with the secondary bodies 9 corresponding to the securing portions 3.

The securing portions 3 extend linearly outwardly away from the main body 8 of the support structure 2. They serve for securing the implant 1 to existing bone structures 10 and, resp., bones, for example by means of screws (not shown), especially osteosynthesis screws, and are configured so that, when abutting on the bone structure 10, they enter into form fit with the latter and constitute so-called bone form-locking portions 11.

In this embodiment, the implant 1 includes three bases 4 formed integrally with the support structure 2. The bases 4 are hollow-cylindrical and have different heights and inclinations. While the implant 1 is usually placed beneath the oral mucosa and, resp., the periosteum, the bases 4 project therefrom into the oral cavity and in this embodiment serve for receiving an intermediate part (not shown) or a so-called abutment (not shown).

Via the intermediate part (not shown) the implant 1 is prepared for holding a prosthesis (not shown) via an abutment (not shown) or the intermediate part (not shown) acts as an abutment (not shown).

FIG. 2 illustrates a spatial view of the implant in a second embodiment, for the mandible. Said second embodiment has, just as the first embodiment, a support structure 2 including plural securing portions 3. In this embodiment, too, the contour of the support structure 2 including the securing portions 3 in advance is exactly adapted to the bone structure 10 on which the implant 1 bears.

The second embodiment includes bases 4 integrally formed with the support structure 2 each of which includes a ball-shaped distal part 12 spaced apart from a truncated cylinder portion 14 of the base 4 via a tapered area 13.

Said distal part 12 may have, instead of being ball-shaped, any other possible geometry such as a frustum-shaped, cylindrical, box-shaped, star-shaped etc. geometry.

The distal part 12 serves as part of a snap-fit connection for receiving/securing the prosthesis (not shown) such as a dental prosthesis, to the implant 1. The prosthesis includes the counter-geometry matching the geometry of the distal part 12.

FIG. 3 shows an enlarged spatial view of the implant 1 of the second embodiment. This figure clearly reveals that the grid-shaped structure 5 or grid structure 5 is exactly adapted to the bone structure 10. The annular portions 6 of the grid-shaped structure 5 are in the form of a through-hole 15 configured to receive a screw (not shown) to be screwed into the bone structure 10, as afore-described.

The second embodiment depicted here has two bases 4 including ball-shaped distal parts 12. The enlarged representation clearly reveals that the two bases 4 have both different heights and different angles of inclination of their central and, resp., longitudinal axes M₁ and M₂ relative to the longitudinal axis L₁ of the implant 1.

The integral formation of the truncated cylinder portion 14 with the distal part 12 spaced apart via the tapered portion 13 allows to realize very small overall heights of the base 4 already which cannot be materialized by a two-part design of the truncated cylinder with an abutment (not shown) adapted to be screwed therein, for example, as a connecting element to the prosthesis by reason of minimum thread lengths.

FIG. 4 illustrates a spatial view of the implant 1 in a third embodiment for the mandible when viewed from the top. This embodiment equally includes two bases 4 that are formed integrally of a truncated cylinder portion 14 and a distal part 12. In contrast to the implant 1 in the second embodiment, the third embodiment shown here has longer securing portions 3 along a direction of the longitudinal axis L₁ of the implant 1 (at the top in this figure).

FIG. 5 illustrates a spatial representation of the implant 1 of the third embodiment for the mandible (from FIG. 4), when viewed from a diagonal lateral direction. This view once again illustrates the accurately fitting configuration of the implant contour and, resp., the support structure 2 including securing portions 3 as a counter-geometry to the bone structure 10 located there beneath and, resp., as bone form-locking portions 11. In other words, the contour of the implant 1 and, resp., of the support structure 2 is exactly adapted to the bone structure 10 on which the implant 1 and, resp., the support structure 2 bears in the secured state.

Said exact shaping assists the operating surgeon in positioning the implant 1 during surgical intervention for inserting the same. In this way, wrong positioning can be avoided and, moreover, additional positioning aids can be largely dispensed with.

The embodiments one to three of the implant 1 can be applied to partially toothed or toothless mandibles so as to compensate for missing teeth by means of a dental prosthesis which is supported by the implant 1.

FIG. 6 illustrates a front view of the implant 1 in a fourth embodiment designed for use in the maxilla. The implant 1 equally includes a support structure 2 having securing portions 3. Said support structure 2 has no grid-shaped structure 5, however (cf. FIG. 1 to FIG. 5). The support structure 2 of this embodiment has plural through-holes 15 in the form of bores including a counterbore portion 16 for receiving e.g. countersunk screws (not shown) via which the implant 1 is connected to the bone structure 10 (not shown here, cf. FIG. 1 to FIG. 5).

Through-holes 15 that are not used for receiving a screw (for securing the implant 1 to the bone structure 10) serve as grow-in areas of bone and soft-tissue structures, thus causing the implant 1 to be so-to-speak “united” with the bone 10 after some time and in this way a tertiary stability to be formed between the bone 10 and the implant 1.

From the support structure 2 three downwardly directed bases 4 are extending which take a hollow-cylindrical shape. Each of the bases 4 has a different height being exactly adjusted to the needs of the respective patient when the implant 1 is designed. The bases 4 are not located on a straight line parallel to the longitudinal axis L₂ of the implant 1 but have a different distance from the longitudinal axis L₂ of the implant 1 (cf. also FIG. 7 in this context).

FIG. 7 shows a top view of the implant 1 of the fourth embodiment for the maxilla. It is clearly evident from this figure that the bases 4 have different distances from the longitudinal axis L₂ of the implant 1: The axes A₃, A₄ and A₅ crossing the center of the bases 4 have different spaces from the longitudinal axis L₂ of the implant 1.

Moreover, the central axes M₃, M₄ and M₅ have different tilt and inclination angles with the longitudinal axis L₂ of the implant 1. Said angles are equally established when the implant is designed so as to optimally adapt the prosthesis received and supported by the implant 1 to the jaw and/or teeth structure of the respective patient. Apart from the optimum alignment of the implant for the chewing stress, this also enables the prosthesis to optimally join esthetically the partial set of teeth.

FIG. 8 illustrates a spatial representation of the implant 1 in a fifth embodiment for the maxilla in the implanted state. In the fifth embodiment, the implant 1 again has a grid-shaped support structure 2. The support structure 2 includes plural securing portions 3 which, as to their contour, are adapted to the existing bone structure 10 (corresponding to the bone form-locking portions 11).

The implant 1 has three bases 4 each of which consists of a hollow-cylindrical truncated cylinder portion 14. In each of the hollow-cylindrical bases 4 an abutment 17 is inserted which consists of a truncated cylinder portion 18 having a ball-shaped distal part 19 formed integrally therewith. For this purpose, the abutment 17 is screwed into the base 4, for example. The ball-shaped distal part 19 serves for connecting the prosthesis (not shown) to the implant 1.

FIG. 9 shows a side view of the implant of the fifth embodiment for the maxilla in the implanted state. This side view reveals that the central axes M₆, M₇ and M₈ of the respective bases 4 again show different tilt and, resp., inclination angles with a longitudinal axis L₃ of the support structure 2. Moreover, the bases 4 may also have slight curvatures as is evident e.g. at the base 4 with the central axis M₆.

The embodiment of the implant 1 shown here is used, for example, to provide toothless maxillae with frameworks or support structures 2 for receiving a prosthesis, with the frameworks interconnecting both sides via the palate. In addition, the implant 1 of the fifth embodiment may also be applied to partially toothed maxillae, wherein the implant 1 is capable of treating states of major bone defects (e.g. in the wake of tumor operations with resection of parts of the maxilla).

### LIST OF REFERENCE NUMERALS

1 implant
2 support structure
3 securing portion
4 base
5 grid-shaped structure
6 annular portion
7 land
8 main body
9 secondary body
10 bone structure
11 bone form-locking portion
12 distal part
13 tapered area
14 truncated cylinder portion
15 through-hole
16 bore including countersunk portion
17 abutment
18 truncated cylinder portion
19 distal part
L₁, L₂, L₃ longitudinal axis
M₁, M₂, M₃, M₄, M₅, M₆, M₇, M₈ central and, resp., longitudinal axis
A₃, A₄, A₅ axis

1. An implant for attaching to a bone with a support structure creating on the basis of individual patient data, the implant comprising: at least one securing portion which follows the bone outer structure and is to be attached to the bone, wherein more bases for receiving a prosthesis directly or by using an intermediate part projects from the support structure, wherein the bases are integral single-piece components of the support structure, and wherein plural bone from-locking portions surrounding the bone are provided and are geometrically configured and aligned so that the form-locking seat on the bone is enforced and so that seat enforces a stable bearing portion of the implant on the bone.

2. The implant according to claim 1, wherein the base is in the form of an elevation or prominence standing out against the ambient outer contour of the support structure.

3. The implant according to claim 1, wherein the base is prepared for non-positively, positively and/or adhesively receiving the prosthesis or an intermediate part.

4-5. (canceled)

6. The implant according to claim 1, further comprising at least three bone form-locking portions spatially separated from each other.

7-8. (canceled)

9. A method for producing an implant according to claim 1, comprising capturing individual patient data by MRT and/or CT, and creating the support structure and/or the base on the basis of the individual patient data based on CAD data.
